(19) United States
(12) Patent Application Publication
(10) Pub. No.: US 2013/0178466 A1
(43) Pub. Date: Jul. 11, 2013

(54) Treatment of Microbial Infections

(71) Applicants: Robin Mark Bannister, London (GB); Wilson Caparros Wanderlay, London (GB); John Brew, London (GB)

(72) Inventors: Robin Mark Bannister, London (GB); Wilson Caparros Wanderlay, London (GB); John Brew, London (GB)

(73) Assignee: BIOCOPEA LIMITED, London (GB)

(21) Appl. No.: 13/509,534

(22) PCT Filed: Nov. 9, 2010
(86) PCT No.: PCT/GB10/51858

§ 371 Date: Jan. 14, 2013

(30) Foreign Application Priority Data
Nov. 11, 2009 (GB) 0919711.2
Feb. 4, 2010 (GB) 1001837.2

(51) Int. Cl. C07D 279/22; A61K 31/404; A61K 31/423; A61K 31/165

(52) U.S. Cl. CPC C07D 279/22; A61K 31/165; A61K 31/404; A61K 31/423
USPC 514/224.8; 564/182; 514/617; 548/500; 514/415; 548/224; 514/375; 544/38

(57) ABSTRACT

The invention provides compositions, medicaments and methods of treating microbial infections, and especially respiratory disorders caused by microbial infections. In particular, the invention relates to the treatment of respiratory diseases caused by pathogenic infections using certain either alkyl substituted or un-substituted 2-aryl acetic acid, or 2-aryl, N-hydroxyacetamide derivatives, or pentoxifylline, and to the use of these compounds in methods of treatment.

This US Non-Provisional patent application is a 35 U.S.C. §371 filing of International Patent Application PCT/GB2010/051858, filed Nov. 9, 2012 which claims priority to GB Patent Application 1001837.2, filed Feb. 4, 2010, and GB Patent Application 0919711.2, filed Nov. 11, 2009, each of which is hereby incorporated by reference in its entirety.

The present invention relates to the treatment of microbial infections, and especially respiratory disorders caused by microbial infections. In particular, the invention relates to the treatment of respiratory diseases caused by pathogenic infections using certain either alkyl substituted or un-substituted 2-aryl acetic acid, or 2-aryl, N-hydroxyacetamide derivatives, or pentoxifylline, and to the use of these compounds in methods of treatment. The invention is particularly concerned with the treatment of viral infections, such as with influenza viral strains, including not only existing viruses, but also future, derivative strains of viruses that have mutated from existing viruses, which could give rise to an influenza pandemic.

Respiratory disease is the term used for diseases of the respiratory system, and includes diseases of the upper and lower respiratory tract, such as the lung, pleural cavity, bronchial tubes, trachea, and of the nerves and muscles that are involved with breathing. Respiratory diseases can be mild and self-limiting, such as the common cold, and so often pass without the need for treatment. However, respiratory disease can also be life-threatening, such as bacterial or viral pneumonia, and so extra care and additional treatment can be required for people who are more vulnerable to the effects of microbial infections, such as the very young, the elderly, people with a pre-existing lung condition, and people with a weakened immune system.

Treatment of respiratory disease depends on the particular disease being treated, the severity of the disease and the patient. Vaccination can prevent certain respiratory diseases, as can the use of antibiotics. However, the growth in viral and fungal infections, and the emergence of antimicrobial drug resistance in human bacterial pathogens, is an increasing problem worldwide. Moreover, since the introduction of antimicrobials, the emergence of resistance has become increasingly prevalent, particularly for important pathogens, such as E. coli and Staphylococcus spp. As a consequence, effective treatment of such micro-organisms and the control of respiratory diseases is becoming a greater challenge.

The defence against disease is critical for the survival of all animals, and the mechanism employed for this purpose is the animal immune system. The immune system is very complex, and involves two main divisions, (i) innate immunity, and (ii) adaptive immunity. The innate immune system includes the cells and mechanisms that defend the host from infection by invading organisms, in a non-specific manner. Leukocytes, which are involved with the innate system, include inter alia phagocytic cells, such as macrophages, neutrophils and dendritic cells. The innate system is fully functional before a pathogen enters the host.

In contrast, the adaptive system is only initiated after the pathogen has entered the host, at which point it develops a defence specific to that pathogen. The cells of the adaptive immune system are called lymphocytes, the two main categories of which are B cells and T Cells. B cells are involved in the creation of neutralising antibodies that circulate in blood plasma and lymph and form part of the humoral immune response. T cells play a role in both the humoral immune response and in cell-mediated immunity. There are several subsets of activator or effector T cells, including cytotoxic T cells (CD8+) and “helper” T cells (CD4+), of which there are two main types known as Type 1 helper T cells (Th1) and Type 2 helper T cell (Th2).

Th1 cells promote a cell-mediated adaptive immune response, which involves the activation of macrophages and stimulates the release of various cytokines, such as IFNγ, TNF-α and IL-12, in response to an antigen. These cytokines influence the function of other cells in the adaptive and innate immune responses, and result in the destruction of micro-organisms. Generally, Th1 responses are more effective against intracellular pathogens, such as viruses and bacteria present inside host cells. A Th2 response, however, is characterised by the release of IL-4, which results in the activation of B cells to make neutralising antibodies, which lead the humoral immunity. Th2 responses are more effective against extracellular pathogens, such as parasites and toxins located outside host cells. Accordingly, the humoral and cell-mediated responses provide quite different mechanisms against an invading pathogen.

The present invention is concerned with the development of novel therapies for the treatment of microbial infections, which cause infections of the respiratory tract. The invention is especially concerned with the development of novel therapies for the treatment of a broad range of viral infections, including acute viral infections, and the treatment of respiratory diseases caused thereby. An acute viral infection is characterized by the rapid onset of disease, a relatively brief period of symptoms, and resolution normally within days. It is usually accompanied by early production of infectious virions and elimination of infection by the host immune system. Acute viral infections are typically observed with pathogens such as influenza virus and rhinovirus. Acute viral infections can be severe, a notable example being the H1N1 influenza virus, which caused the 1918 Spanish flu pandemic.

Acute infections begin with an incubation period, during which the viral genomes replicate and the host innate responses are initiated. The cytokines produced early in infection lead to classical symptoms of an acute infection: aches, pains, fever, and nausea. Some incubation periods are as short as 1 day (influenza, rhinovirus), indicating that the symptoms are produced by local viral multiplication near the site of entry. An example of a classic acute infection is uncomplicated influenza. Virus particles are inhaled in droplets produced by sneezing or coughing, and begin replicating in ciliated columnar epithelial cells of the respiratory tract. As new infectious virions are produced, they spread to neighboring cells. Virus can be isolated from throat swabs or nasal secretions from day 1 to day 7 after infection. Within 48 hours after infection symptoms appear, and these last about 3 days and then subside. The infection is usually cleared by the innate and adaptive responses in about 7 days. However, the patient usually feels unwell for several weeks, a consequence of the damage to the respiratory epithelium by the cytokines produced during infection.

Acute viral infections, such as influenza and measles, are responsible for epidemics of disease involving millions of individuals each year. When vaccines are not available, acute infections are difficult to control. This makes it exceedingly difficult to control acute infections in large populations and crowded areas. The frequent outbreak of norovirus gastroenteritis, a classic acute infection, highlights the problem. Antiviral therapy cannot be used, because it must be given early in infection to be effective. There is thus little hope of treating most acute viral infections with antiviral drugs until rapid diagnostic tests become available. However, it should be noted that there are currently no antivirals for most common acute viral diseases. There is, therefore, clearly a need in the art for improved medicaments for use in the treatment of viral infections, and especially acute viral infections.

The inventors have determined that certain either alkyl substituted or un-substituted 2-aryl acetic acid, or 2-aryl, N-hydroxyacetamide derivatives have the properties to be useful in treating microbial infections.

Thus, according to a first aspect of the invention, there is provided a compound of formula I:—

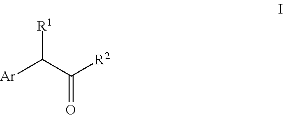

wherein, Ar is an aryl or substituted aryl group, R¹ is a C₁₋₃ alkyl group or hydrogen, and R² is OH or —NHOH, or a pharmaceutically acceptable salt, solvate, or solvate of a salt thereof, for use in treating an infection with a pathogen, which causes a respiratory disorder.

The compound of formula I may be used to treat an infection with a pathogen which causes a fulminant respiratory disorder. In one embodiment, the compound of formula I may be used to treat a viral infection, preferably an acute viral infection.

Ar is preferably a substituted phenyl group. R¹ is preferably hydrogen or a methyl group. R² is preferably —NHOH.

When Ar is a substituted phenyl group, it is preferred for the bond joining it to the remainder of the structure shown in formula I to extend directly to a carbon atom in the phenyl ring.

In the context of the invention, the term “aryl” refers to groups derived from arenes or heteroarenes.

The preferred compounds according to the invention are 2-aryl, N-hydroxyacetamide, or 2-aryl, 2-methyl, N-hydroxyacetamide derivatives.

Specific embodiments of compounds useful in the present invention include the following:—

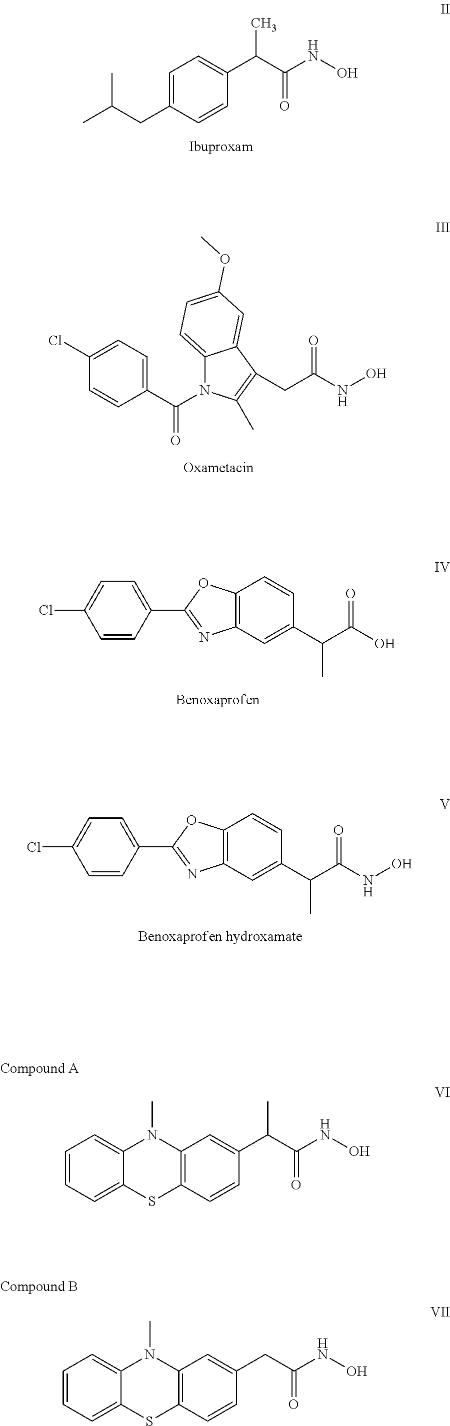 [SMILES: CC(C)Cc1ccc(C(C)C(=O)NO)cc1 ; COc1ccc2c(c1)c(CC(=O)NO)c(C)n2C(=O)c1ccc(Cl)cc1 ; CC(C(=O)O)c1ccc2oc(-c3ccc(Cl)cc3)nc2c1 ; CC(C(=O)NO)c1ccc2oc(-c3ccc(Cl)cc3)nc2c1 ; CC(C(=O)NO)c1ccc2c(c1)N(C)c1ccccc1S2 ; CN1c2ccccc2Sc2ccc(CC(=O)NO)cc21]

The aforementioned specific compounds can each also be used in the form of a pharmaceutically acceptable salt, solvate, or solvate of a salt.

Certain compounds in accordance with the invention are chiral. The invention therefore encompasses the use of such compounds in the form of racemic mixtures, enantiomerically enriched mixtures, or as substantially pure enantiomers. The compounds of the present invention can be obtained from commercial sources or manufactured using standard synthetic procedures.

In a second aspect, there is provided pentoxifylline, or a pharmaceutically acceptable salt, solvate, or solvate of a salt thereof, for use in treating an infection with a pathogen, which causes a respiratory disorder.

Pentoxifylline, or a pharmaceutically acceptable salt, solvate, or solvate of a salt thereof may be used to treat an infection with a pathogen, which causes a fulminant respiratory disorder. For example, pentoxifylline, or a pharmaceutically acceptable salt, solvate, or solvate of a salt thereof, may be used to treat a viral infection, preferably an acute viral infection.

Thus, in another aspect, the present invention relates to the treatment of viral infections using pentoxifylline, or a pharmaceutically acceptable salt, solvate, or solvate of a salt thereof.

It is known that, during an acute viral infection, such as influenza, the virus is predominantly fought through the host's innate immune system and the cell-mediated, Th1 response, and subsequently by the humoral, antibody-driven Th2 response. Furthermore, the inventors believe that, in susceptible individuals (i.e. the young, and fit and healthy individuals), the Th1 response to an influenza infection can be extremely strong, and can give rise to a so-called “cytokine storm”, involving a significant increase in the concentration of certain cytokines, such as IFN-γ and TNF-α. This “cytokine storm” can result in serious inflammation of infected lung tissue, the leakage of fluid into the lungs and significant damage to the lungs of an infected individual. The end result can be a respiratory disorder, such as pulmonary oedema or a secondary bacterial infection, which can eventually kill the infected individual, rather than the virus itself.

Baumgarth and Kelso (J. Virol., 1996, 70, 4411-4418) reported that neutralisation of the Th1 cytokine, IFN-γ, can lead to a significant reduction in the magnitude of the cellular infiltrate in lung tissue following infection, and suggested that IFN-γ may be involved in the mechanisms that regulate increased leukocyte traffic in the inflamed lung. They also postulated that IFN-γ affects the local cellular response in the respiratory tract, as well as the systemic humoral response to influenza virus infection. Based on the findings of this study, the inventors of the present invention considered whether suppression of the cytokines, IFN-γ and TNF-α, may be useful for treating influenza.

As described in the Examples, the inventors studied the in vitro effects of alkyl substituted or un-substituted 2-aryl acetic acid, or 2-aryl, N-hydroxyacetamide derivatives or pentoxifylline, on blood cells that had been stimulated in such a way that they reflected an acute viral infection. As a model of viral infection, they used blood cell samples that had been stimulated with mitogens (lipopolysaccharide or Concanavalin A), compounds that trigger signal transduction pathways, and which thereby stimulate lymphocytes present in the blood sample to commence mitosis. This model therefore closely replicates the processes that are induced by a viral infection, and enables the direct assessment of the immune response exhibited by the lymphocytes upon treatment with the test compounds.

As described in the examples, the inventors found, using this in vitro model, that ibuproxam, benoxaprofen hydroxamate or pentoxifylline effectively inhibited the production of both of the cytokines, IFN-γ and TNF-α. Thus, the invention is based on the control of the Th1 immune system, which is driven by IFN-γ, and which is responsible for the hyperimmune cell-mediated response that causes respiratory collapse in susceptible individuals (e.g. the young and healthy).

These compounds are representative of a family of active compounds that share a common alkyl substituted or un-substituted 2-aryl acetic acid, or 2-aryl, N-hydroxyacetamide derivatives core structure or pentoxifylline and which are known to exhibit similar physiological activities. This family of compounds is defined by formula (I) and it follows, because they all share the same activity providing motif, that they can all be effectively used to prevent IFN-γ and TNF-α levels from rising in the “cytokine storm” following a viral infection.

As described in the Examples, the inventors have also demonstrated, in an in vivo mouse model, that the compounds described herein may be used to prevent, treat or ameliorate respiratory diseases caused by viral infections. The inventors therefore believe that they are the first to demonstrate that, in addition to sharing other properties, the defined either alkyl substituted or un-substituted 2-aryl acetic acid, or 2-aryl, N-hydroxyacetamide derivatives or pentoxifylline can be used to modulate TNF-α and IFN-γ in such a way so as to be useful in the treatment of acute and chronic viral infections.

A common pathogen-induced respiratory disorder or acute respiratory distress, is hospital- and community-acquired pneumonia. Pneumonia is characterised by cough, chest pains, fever, and difficulty in breathing due to pulmonary oedema. These symptoms occur in all pneumonia patients regardless of the pathogen that causes the pneumonia, which can be bacterial (e.g. Streptococcus pneumonia), viral (e.g. influenza virus) and fungal (e.g. Histoplasma capsulatum). Regardless of the pathogen causing pneumonia, the symptoms are the same and the inflammatory processes, regardless of the stimulus, cause exaggerated inflammatory responses, resulting in potentially fatal pulmonary oedema. In animal models of respiratory disorders associated with the influenza infection (i.e. a viral pathogen), the end points are designed to measure pulmonary oedema related end points (i.e. post infection survival). The effect on post infection survival for the compounds described herein, in the influenza assay, supports the likelihood for effects in pulmonary oedema caused by any type of pathogen, be it viral, bacterial or fungal.

Accordingly, the inventors believe that these compounds may be used to combat respiratory disorders that are caused by any microbial or pathogenic infection, such as bacterial, viral (e.g. acute viral infections) or fungal, and which, in some cases (e.g. influenza infections), can cause death.

Various metabolites of the invention may also be used for treating microbial infections. Compound (I), for use, in the invention, may be chiral. Hence, the compound may include any diastereomer and enantiomer. Diastereomers or enantiomers are believed to display potent cytokine modulatory activity, and such activities may be determined by use of appropriate in vitro and in vivo assays, which will be known to the skilled technician. It will also be appreciated that compounds for use in the invention may also include pharmaceutically active salts, solvates or solvates of a salt, e.g. the hydrochloride.

Furthermore, in a third aspect of the invention, there is provided a method of preventing, treating and/or ameliorating a microbial infection, the method comprising administering, to a subject in need of such treatment, a therapeutically effective amount of a compound as previously defined.

The inventors have demonstrated that the compounds of the invention may be used in the treatment of any number of microbial infections, and respiratory disorders which may result therefrom, such as pneumonia. The compounds may be used as a prophylactic (to prevent the development of a respiratory disorders associated with microbial infection), or they may be used to treat existing respiratory disorders associated with microbial infections. Thus, the compounds described herein are of utility as compositions for the treatment of respiratory disorder associated microbial infections.

Examples of micro-organisms, which may cause a respiratory disorder, which may be treated with compounds according to the invention, may include bacteria, viruses, fungi, or protozoa, and any other pathogens and parasites, which cause respiratory disorders. These pathogens can cause upper or lower respiratory tract diseases, or obstructive or restrictive lung diseases, each of which may be treated. The most common upper respiratory tract infection is the common cold, which may be treated. In addition, infections of specific organs of the upper respiratory tract, such as sinusitis, tonsillitis, otitis media, pharyngitis and laryngitis are also considered as upper respiratory tract infections, which may be treated with the compounds described herein.

The most common lower respiratory tract infection is pneumonia, which may be treated with the compounds described herein. Pneumonia is usually caused by bacteria, particularly Streptococcus pneumoniae. However, tuberculosis is also an important cause of pneumonia. Other pathogens, such as viruses and fungi, can also cause pneumonia, for example Severe Acute Respiratory Distress, Acute Respiratory Distress Syndrome and pneumocystis pneumonia. Therefore, the compounds of the invention may be used to treat Respiratory Distress Syndrome (RDS), Acute Respiratory Distress Syndrome (ARDS), or Acute Lung Injury (ALI). In addition the compounds may be used to treat diseases with concomitant pathogen infection such as chronic obstructive pulmonary disorder, cystic fibrosis and bronchiolitis.

The method of the third aspect may be useful for preventing, treating and/or ameliorating a respiratory disorder caused by a bacterial infection. In particular, the compounds described herein may be used for the treatment of a variety of respiratory bacterial infections, including bronchopulmonary infections, for example pneumonia; or ear, nose, and throat infections, for example otitis media, sinusitis, laryngitis and diphtheria.

The bacterium causing the infection may be a Gram-positive bacterium or a Gram-negative bacterium. Examples of bacteria, which may cause a respiratory disorder, against which the compounds in accordance with the invention are effective, may be selected from a list consisting of: Streptoccoccus spp., Staphylococcus spp., Haemophilus spp., Klebsiella spp., Escherichia spp., Pseudomonas spp., Moraxella spp., Coxiella spp., Chlamydophila spp., Mycoplasma spp., Legionella spp. and Chlamydia spp.

Species of bacteria, which may cause a respiratory disorder, against which the compositions in accordance with the invention are effective, may be selected from a list consisting of: Streptoccoccus pneumoniae, Staphylococcus aureus, Haemophilus influenzae, Klebsiella pneumoniae, Escherichia coli, Pseudomonas aeroginosa, Moraxella catarrhalis, Coxiella burnettie, Chlamydophila pneumoniae, Mycoplasma pneumoniae, Legionella pneumophila and Chlamydia trachomatis.

The method of the third aspect may be useful for preventing, treating and/or ameliorating a fungal infection. The compounds described herein may be used for the treatment of a variety of fungal infections and disease conditions, including bronchopulmonary infections, for example pneumonia.

Examples of fungi, which may cause a respiratory disorder, against which the compositions in accordance with the invention are effective, may be selected from a group consisting of: Histoplasma spp., Blastomyces spp., Coccidioides spp., Cryptococcus spp., Pneumocystis spp. and Aspergillus spp.

Species of fungi, which may cause a respiratory disorder, against which the compositions in accordance with the invention are effective, may be selected from a group consisting of: Histoplasma capsulatum, Blastomyces dermatitidis, Coccidioides immitis, Cryptococcus neoformans, Pneumocystis jiroveci, Aspergillus flavus, Aspergillus fumigatus, Aspergillus nidulans, Aspergillus niger, Aspergillus parasiticus and Aspergillus terreus.

The method of the third aspect may be particularly useful for preventing, treating and/or ameliorating a viral infection. The compounds described herein may be used for the treatment of a variety of viral infections, including bronchopulmonary infections, for example pneumonia.

The inventors believe that the compounds of the invention may be used in the treatment of any number of acute or chronic viral infections, and respiratory disorders which may result therefrom. The compounds may be used as a prophylactic (to prevent the development of a viral infection) or may be used to treat existing viral infections. The virus may be any virus, and may be an enveloped virus. The virus may be an RNA virus or a retrovirus. For example, the viral infection, which may be treated, may be a paramyxovirus or an orthomyxovirus infection. The virus causing the infection may be a poxvirus, iridovirus, thogavirus, or torovirus. The virus causing the infection may be a filovirus, arenavirus, bunyavirus, or a rhabdovirus. It is envisaged that the virus may be a hepadnavirus, coronavirus, or a flavivirus.

In particular, the following viral infections linked to respiratory complications may be treated: Respiratory syncytial virus, Human bocavirus, Human parvovirus B19, Herpes simplex virus 1, Varicella virus, Adenovirus, Parainfluenza virus, Enterovirus 71, Hantavirus, SARS virus, SARS-associated coronavirus, Sin Nombre virus, Respiratory reovirus, Haemophilus influenza or Adenovirus.

The invention extends to the treatment of infections with derivatives of any of the viruses disclosed herein. The term “derivative of a virus” can refer to a strain of virus that has mutated from an existing viral strain.

The virus may be selected from the group of viral genera consisting of Influenzavirus A; Influenzavirus B; Influenzavirus C; Isavirus and Thogotovirus, or any derivative of the foregoing viruses. Influenza viruses A-C include viruses that cause influenza in vertebrates, including birds (i.e. avian influenza), humans, and other mammals. Influenzavirus A causes all flu pandemics and infects humans, other mammals and birds. Influenzavirus B infects humans and seals, and Influenzavirus C infects humans and pigs. Isaviruses infect salmon, and thogotoviruses infect vertebrates (including human) and invertebrates.

Thus, compounds of the invention may be used to treat an infection of any of Influenzavirus A, Influenzavirus B, or Influenzavirus C, or a derivative thereof. It is preferred that the compound may be used for treating an infection of Influenza A, or a derivative thereof. Influenza A viruses are classified, based on the viral surface proteins hemagglutinin (HA or H) and neuraminidase (NA or N). Sixteen H subtypes (or serotypes) and nine N subtypes of Influenza A virus have been identified. Thus, the compounds of the invention may be used to treat an infection of any serotype of Influenzavirus A selected from the group of serotypes consisting of: H1N1; H1N2; H2N2; H3N1; H3N2; H3N8; H5N1; H5N2; H5N3; H5N8; H5N9; H7N1; H7N2; H7N3; H7N4; H7N7; H9N2; and H10N7, or a derivative thereof. The inventors believe that compounds of the invention may be particularly useful for treating viral infections of H1N1 virus, or a derivative thereof. It will be appreciated that swine flu is a strain of the H1N1 virus.

The inventors have found that, following infection with a virus, IFN-γ and TNF-α can cause fluid to leak into the lungs of an infected subject, which results in respiratory disorders that can cause eventual death. Although they do not wish to be bound by hypothesis, the inventors believe that the compounds of the invention may be used to treat viral infections because they can act as an inhibitor of cytokine production, and in particular IFN-γ and TNF-α, and that, therefore, they can be used to treat the respiratory disorder caused by a viral infection.

The compounds of the invention may therefore be used to ameliorate inflammatory symptoms of virally induced cytokine production. The anti-inflammatory compound may have an effect on any cytokine. However, preferably it modulates IFN-γ and/or TNF-α. The compounds may be used to treat inflammation in an acute viral infection of a naïve subject. The term “naïve subject” can refer to an individual who has not previously been infected with the virus. It will be appreciated that once an individual has been infected with a virus such as herpes, that individual will always retain the infection.

It is especially intended that the compounds may be used to treat the final stages of a viral infection, such as the end stages of influenza. The compound represented by formula I or pentoxifylline may also be used to treat a viral flare-up. A viral flare-up can refer to either the recurrence of disease symptoms, or an onset of more severe symptoms.

It will be appreciated that the compound of formula (I) or pentoxifylline may be used to treat microbial (e.g. viral) infections in a monotherapy (i.e. use of the compound (I) alone). Alternatively, the compounds of the invention may be used as an adjunct to, or in combination with, known antimicrobial therapies. For example, conventional antibiotics for combating bacterial infections include amikacin, amoxicillin, aztreonam, cefazolin, cefepime, ceftazidime, ciprofloxacin, gentamicin, imipenem, linezolid, nafcillin, piperacillin, quinopristin-dalfoprisin, ticarcillin, tobramycin, and vancomycin. In addition, compounds used in antiviral therapy include acyclovir, gangcylovir, ribavirin, interferon, nucleotide or non-nucleoside inhibitors of reverse transcriptase, protease inhibitors and fusion inhibitors. Furthermore, conventional antifungal agents include, for example farnesol, clotrimazole, ketoconazole, econazole, fluconazole, calcium or zinc undecylenate, undecylenic acid, butenafine hydrochloride, ciclopirox olaimine, miconazole nitrate, nystatin, sulconazole, and terbinafine hydrochloride. Hence, compounds according to the invention may be used in combination with such antibacterial, antiviral and antifungal agents.

The compound of the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle for medicaments according to the invention should be one which is well tolerated by the subject to whom it is given, and preferably enables delivery of the agents across the blood-brain barrier, or directly to the site infected by the pathogen (i.e. the virus, bacterium or fungus), such as the lungs, in order to treat the respiratory disease.

Compositions comprising the compounds of the invention may be used in a number of ways. For instance, oral administration may be required in which case the compound may be contained within a composition that may, for example, be ingested orally in the form of a tablet, capsule or liquid. Alternatively, the composition may be administered by injection into the blood stream. Injections may be intravenous (bolus or infusion) or subcutaneous (bolus or infusion). Alternatively, the composition comprising the compounds of the invention may be administered by inhalation (e.g. intranasally, or by mouth) or rectally (e.g. suppository).

Compositions may also be formulated for topical use. For instance, ointments may be applied to the skin, areas in and around the mouth or genitals to treat specific viral infections. Topical application to the skin is particularly useful for treating viral infections of the skin or as a means of transdermal delivery to other tissues.

It will be appreciated that the amount of compound that is required is determined by its biological activity and bioavailability, which in turn depends on the mode of administration, the physicochemical properties of the compound and whether the compound is being used as a monotherapy, or in a combined therapy. The frequency of administration will also be influenced by the above-mentioned factors and particularly the half-life of compounds within the subject being treated.

Optimal dosages to be administered may be determined by those skilled in the art, and will vary with the particular compound in use, the strength of the preparation, the mode of administration, and the advancement of the disease condition. Additional factors depending on the particular subject being treated will result in a need to adjust dosages, including subject age, weight, gender, diet, and time of administration.

It will be appreciated that a skilled person will be able to calculate required doses, and optimal concentrations of compound (I) and pentoxifylline at a target tissue, based upon the pharmacokinetics of the chosen compound. Known procedures, such as those conventionally employed by the pharmaceutical industry (eg in vivo experimentation, clinical trials, etc.), may be used to establish specific formulations of the compounds of the invention and precise therapeutic regimes (such as daily doses of the compounds and the frequency of administration).

Generally, a daily dose of between 0.001 μg/kg of body weight and 20 mg/kg of body weight of the compounds may be used for the prevention and/or treatment of a microbial (e.g. viral) infection depending upon which compound is used. Suitably, the daily dose is between 0.01 μg/kg of body weight and 10 mg/kg of body weight, more suitably between 0.01 μg/kg of body weight and 1 mg/kg of body weight or between 0.1 μg/kg and 100 μg/kg body weight, and most suitably between approximately 0.1 μg/kg and 10 μg/kg body weight.

Daily doses of the compounds may be given as a single administration (e.g. a single daily injection or a single inhalation). A suitable daily dose may be between 0.07 μg and 700 mg (i.e. assuming a body weight of 70 kg), or between 0.70 μg and 500 mg, or between 10 mg and 450 mg. The medicament may be administered before or after infection with the pathogen causing the respiratory disorder, such as the virus. The medicament may be administered within 2, 4, 6, 8, 10 or 12 hours after infection. The medicament may be administered within 14, 16, 18, 20, 22, or 24 hours after infection. The medicament may be administered within 1, 2, 3, 4, 5, or 6 days after infection, or at any time period therebetween.

In embodiments where the infection being treated is an infection of influenza, independently of whether or not the influenza is a pandemic influenza, the subject is someone treated with medicaments comprising the compounds of the invention in whom symptoms of respiratory difficulty arise and/or in whom cytokine levels (any of the above mentioned cytokines, but typically IFN-α, or TNF-γ) increase at the onset of symptoms of respiratory difficulty. More preferably, the subject is a subject in whom symptoms of respiratory difficulty arise, and/or in whom cytokine levels increase, at the following times after onset of influenza symptoms: from 12, 24, 18 or 36 hours or more (more preferably from 48 hours or more, from 60 hours or more, or from 72 hours or more; most preferably from 36-96 hours, from 48-96 hours, from 60-96 hours or from 72-96 hours). Alternatively, and independently of whether or not the influenza is a pandemic influenza, the subject is someone in whom symptoms of respiratory difficulty arise and/or in whom cytokine levels increase, at the onset (or early stage) of recruitment of the adaptive immune system into the infected lung.

As described in the in vivo mouse studies in the Examples, the inventors have shown that mice that were administered with more than one dose of a cytokine inhibitor showed improvement to symptoms of the influenza infection. Therefore, it is envisaged that medicaments comprising compound (I) or pentoxifylline may be administered more than once to the subject in need of treatment. The compound may require administration twice or more times during a day. As an example, compound (I) may be administered as two (or more depending upon the severity of the viral infection being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter, and so on. It is envisaged that the compound may be administered every day (more than once if necessary) following viral infection.

Thus, the compounds of the invention are preferably suitable for administration to a subject as described above, preferably suitable for administration at the aforementioned points after the onset of influenza symptoms.

Alternatively, a slow release device may be used to provide optimal doses of compounds according to the invention to a patient without the need to administer repeated doses.

Based on their findings that the compounds described herein may be used to reduce the levels of cytokines, such as TNF-α and IFN-γ, the inventors believe that these effects of the compounds may be harnessed and used in the manufacture of clinically useful compositions.

Hence, in a fourth aspect, there is provided a pharmaceutical composition comprising a therapeutically effective amount of a compound represented by the general formula I or pentoxifylline, as previously defined, and a pharmaceutically acceptable vehicle, for use in the treatment of an infection with a pathogen, which causes a respiratory disorder.

The infection may be acute or chronic.

A “therapeutically effective amount” of a compound represented by formula (I) or pentoxifylline is any amount which, when administered to a subject, results in decreased levels of cytokines, such as TNF-α and IFN-γ, and thereby provides prevention and/or treatment of a microbial infection, such as an acute viral infection.

For example, the therapeutically effective amount of compound (I) or pentoxifylline used may be from about 0.07 μg to about 700 mg, and preferably from about 0.7 μg to about 70 mg. The amount of compound (I) is from about 7 μg to about 7 mg, or from about 7 μg to about 700 μg.

A “subject” can be a vertebrate, mammal, or domestic animal, and is preferably a human being. Hence, medicaments according to the invention may be used to treat any mammal, for example human, livestock, pets, or may be used in other veterinary applications.

A “pharmaceutically acceptable vehicle” as referred to herein can be any combination of known compounds known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agent (i.e. the compound (I) or pentoxifylline according to the invention). In tablets, the active agent may be mixed with a vehicle having the necessary compression properties in suitable proportions and compacted in the shape and size desired. The powders and tablets preferably contain up to 99% of the active agent. Suitable solid vehicles include, for example calcium phosphate, magnesium stearate, talc, sugars, lactose, dextrin, starch, gelatin, cellulose, polyvinylpyrrolidine, low melting waxes and ion exchange resins.

In another embodiment, the pharmaceutical vehicle may be a gel and the composition may be in the form of a cream or the like. In yet another embodiment, the pharmaceutical vehicle may be a liquid, and the pharmaceutical composition may be in the form of a solution. Liquid vehicles are used in preparing solutions, suspensions, emulsions, syrups, elixirs and pressurized compositions. The active compound may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellant.

Liquid pharmaceutical compositions which are sterile solutions or suspensions can be utilized by, for example, intramuscular, intrathecal, epidural, intraperitoneal, intravenous and particularly subcutaneous injection. The compound according to the invention may be prepared as a sterile solid composition that may be dissolved or suspended at the time of administration using sterile water, saline, or other appropriate sterile injectable medium.

The compound may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compound can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

All of the features described herein (including any accompanying claims, abstract and drawings), and/or all of the steps of any method or process so disclosed, may be combined with any of the above aspects in any combination, except combinations where at least some of such features and/or steps are mutually exclusive.

Embodiments of the invention will now be further described, by way of example only, with reference to the following Examples, and to the accompanying diagrammatic drawings, in which:—

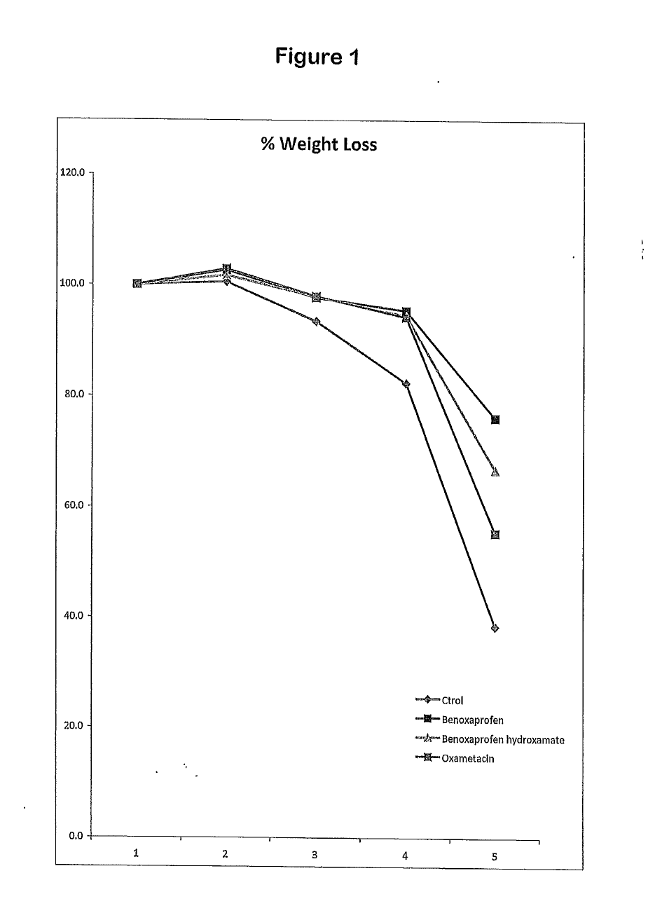

FIG. 1 is a graph showing the results of an in vivo mouse challenge, in which mice were infected with a H1N1 virus, and then treated with a compound represented by formula I, i.e. benoxaprofen (BC1005), benoxaprofen hydroxamate (BC1006) or oxametacin (BC1002). Benoxaprofen, benoxaprofen hydroxamate or oxametacin was administered to the mice as a single dose on day 3 and the weight loss of the mice was measured. No benoxaprofen, benoxaprofen hydroxamate or oxametacin was added to the control mice;

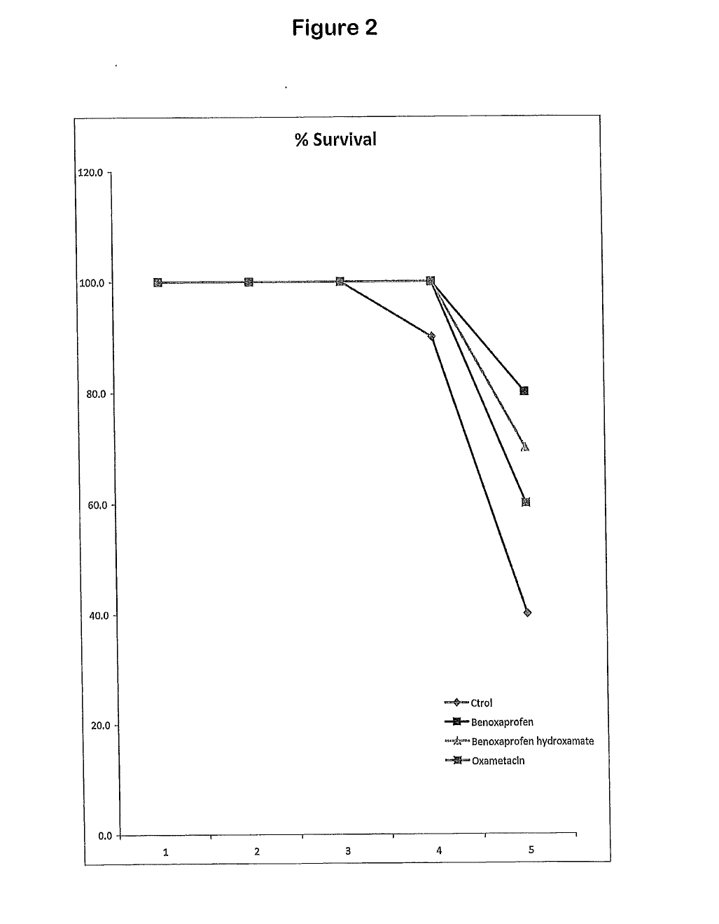

FIG. 2 is a graph showing the survival rate of mice in the in vivo mouse challenge described in relation to FIG. 1. The mice were administered with benoxaprofen (BC1005), benoxaprofen hydroxamate (BC1006) or oxametacin (BC1002) as a single dose on day 3 and the percentage rate of survival was measured. No benoxaprofen, benoxaprofen hydroxamate or oxametacin was added to the mice of the control;

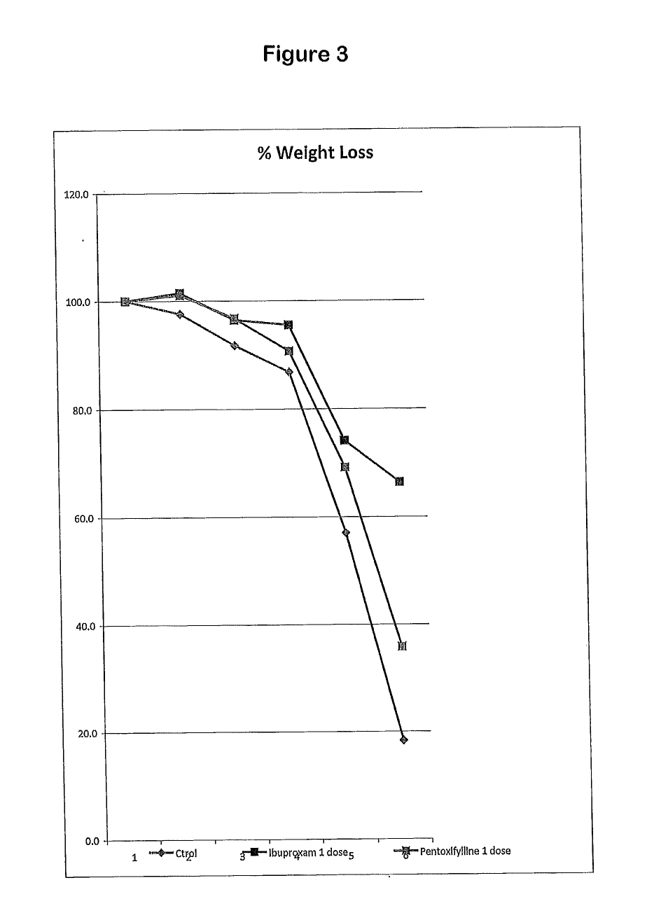

FIG. 3 is a graph showing the results of an in vivo mouse challenge, in which mice were infected with a H1N1 virus, and then treated with a compound represented by formula I, i.e. ibuproxam (BC1048), or pentoxifylline (BC1042). Ibuproxam or pentoxifylline was administered to the mice as a single dose on day 3, and the weight loss of the mice was measured. No ibuproxam or pentoxifylline was added to the control mice;

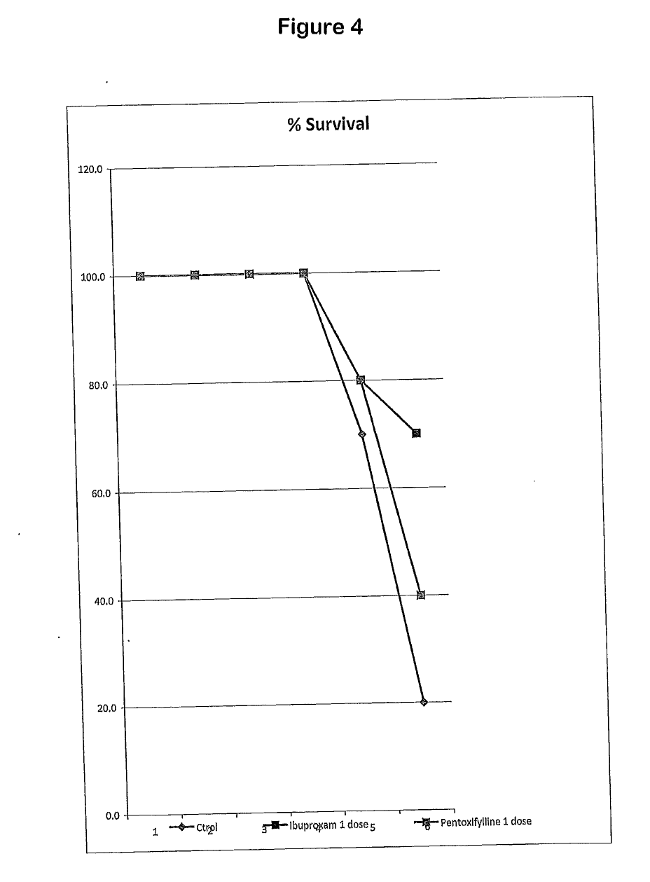

FIG. 4 is a graph showing the survival rate of mice in the in vivo mouse challenge described in relation to FIG. 3. The mice were administered with ibuproxam or pentoxifylline as a single dose on day 3, and the percentage rate of survival was measured. No ibuproxam or pentoxifylline was added to the mice of the control;

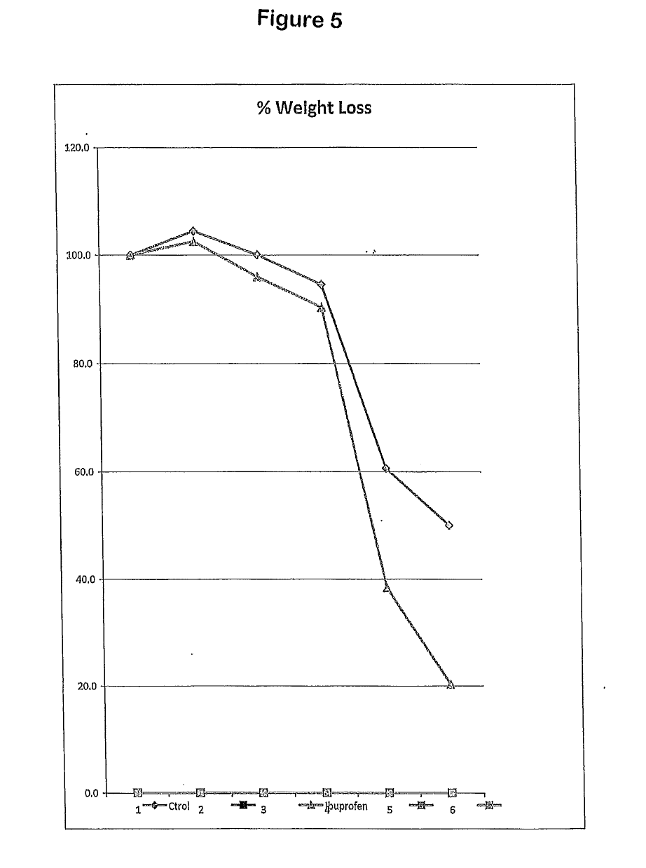

FIG. 5 is a graph showing the results of an in vivo mouse challenge, in which mice were infected with a H1N1 virus, and then treated with a compound represented by formula I, i.e. ibuproxam. Ibuproxam was administered to the mice as a single dose on day 3 and the weight loss of the mice was measured. No ibuproxam was added to the control mice and instead ibuprofen was administered to these mice as a comparison compound; and

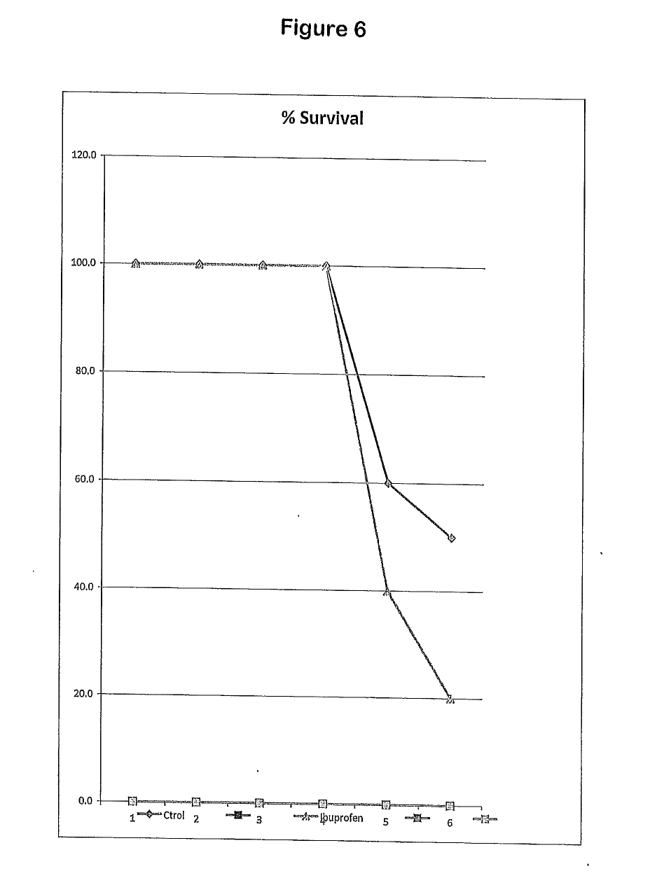

FIG. 6 is a graph showing the survival rate of mice in the in vivo mouse challenge described in relation to FIG. 5. The mice were administered with ibuproxam as a single dose on day 3 and the percentage rate of survival was measured. No ibuproxam was added to the mice of the control and instead ibuprofen was administered to these mice as a comparison compound.

### EXAMPLES

The inventors carried out a range of in vitro and in vivo experiments in order to determine the effects of various compounds represented by formula I or pentoxifylline on the production of the cytokines, IFN-γ and TNF-α. The inventors have demonstrated in the results described below that the compounds of the invention surprisingly act as inhibitors of IFN-γ and TNF-α. Furthermore, they have demonstrated in in vivo mouse models that administration of said compounds results in a reduction in the viral symptoms (i.e. reduction in weight loss, increase in survival rate, and reduction in total morbidity) in mice.

### Materials and Methods

### In Vivo Mouse Studies

Protocol: Fifty (50) C57BL/6 female mice (6-7 weeks old), were divided into five (5) experimental groups containing ten (10) animals each. On day 1, animals received an intranasal lethal dose (50 μl total, 25 μl nostril) of Influenza A/PR/8/34 under halothane induced anaesthesia. On Day 3, animals received one intra-peritoneal injection (100-150 μl) of the test compound (360 μg ibuproxam (BC1048), 54 μg oxametacin (BC1002), 180 μg benoxaprofen (BC 1005), 189 μg benoxaprofen hydroxamate (BC1006)).

All animals were assessed daily for morbidity, weight loss and survival from Day 1 until at least Day 6. Morbidity variables (i.e. Body Condition, Posture, Activity, Piloerection, Respiration, Vocalisation, Ataxia and Oculo/Nasal Discharges) were recorded according to the following scale of severity: Normal (0), Mild (1), Laboured (2) and Severe/Cull-point (3).

### Examples

### In Vivo Mouse Studies

Using standard techniques as described above, mice were infected with a H1N1 virus which was allowed to become established in each of the subjects. Each test mouse was then treated with ibuproxam (BC1048), oxametacin (BC1002), benoxaprofen (BC1005), benoxaprofen hydroxamate (BC1006) or pentoxifylline with a single dose on day 3 after infection with the virus. In the control mice, no ibuproxam (BC1048), oxametacin (BC1002), benoxaprofen (BC 1005), benoxaprofen hydroxamate (BC1006) or pentoxifylline (BC1042) was administered. The weight loss of both treated and untreated mice was then determined.

As shown in FIGS. 1, 3 and 5 the mice that received doses of ibuproxam (BC1048), oxametacin (BC1002), benoxaprofen (BC 1005), benoxaprofen hydroxamate (BC1006) or pentoxifylline (BC1042) showed at least a 10% lower reduction in weight loss than the control mice. Accordingly, although the inventors do not wish to be bound by hypothesis, they believe that the reduced levels of the cytokines, IFN-γ and TNF-α, in H1N1-infected mice upon exposure to ibuproxam, oxametacin, benoxaprofen, benoxaprofen hydroxamate or pentoxifylline results in the mice maintaining their weight.

Referring to FIGS. 2, 4 and 6, there are shown the results of percentage survival of mice treated with ibuproxam, oxametacin, benoxaprofen, benoxaprofen hydroxamate or pentoxifylline. As can be seen, mice treated with ibuproxam, oxametacin, benoxaprofen, benoxaprofen hydroxamate or pentoxifylline, showed a higher survival rate than the control, untreated mice.

### In Vitro Studies—Stimulation Experiments Using Mitogens, LPS and Con A

Plasma B cells can enter mitosis when they encounter an antigen matching their immunoglobulin. A mitogen is a chemical substance that triggers signal transduction pathways in which mitogen-activated protein kinase is involved, thereby encouraging a cell to commence cell division, leading to mitosis. Thus, mitogens can be effectively used to stimulate lymphocytes and therefore assess immune function. By stimulating lymphocytes, mitogens can be used to replicate the effects of a viral infection.

The two mitogens that the inventors used to stimulate lymphocytes, and therefore assess immune function, were lipopolysaccharide (LPS) and Concanavalin A (Con A). LPS acts on B cells but not T cells, whereas Con A acts on T cells but not B cells. The effects of two embodiments of the compound represented by formula I, i.e. ibuproxam (referred to in the tables as BC1048) and benoxaprofen hydroxamate (BC1006), and pentoxifylline (referred to in the tables as BC1042) on the levels of IFN-γ and TNF-α were investigated in LPS and Con A stimulated assays. Peripheral Blood Mononuclear Cells (PMBC) were independently administered with each mitogen, LPS or Con A, and then treated with ibuproxam, benoxaprofen hydroxamate or pentoxifylline. Control experiments were conducted in which no LPS or Con A was added, such that any effect on the levels of IFN-γ and TNF-α could be directly attributed to the presence of the test compound, ibuproxam, benoxaprofen hydroxamate or pentoxifylline.

### Materials and Methods

### Isolation, Culture and Treatment of Peripheral Blood Mononuclear Cells (PBMC)

Blood was collected in 6 ml vacutainers (green cap). Blood was processed within 2 h of collection.

Materials used: Non-coagulated blood; FCS; RPMI-1640 media supplemented with L-Gln and P/S; PBS; sterile tips and pipettes; Sterile 15 ml Falcon; Sterile V-bottom 96-well plates with lids; Neubauer chamber; Trypan Blue solution; 70% IPA solution; Accuspin-Histopaque tubes (Sigma, A7054)

Procedure:
1. Dilute samples 1:1 in sterile PBS;
2. Add 30 ml of diluted blood into an Accuspin-Histopaque tube (Sigma, A7054);
3. Centrifuge at 800 rcf 15 min at room temperature (RT);
4. After centrifugation, the red blood cells will remain at the bottom below the frit. The monocytes (PBMC) will be present on a layer above the frit, with the plasma on top;
5. Collect the PBMC layer with a pipette into a fresh 15 ml Falcon tube and top up to 15 ml of PBS;
6. Centrifuge at 250 rcf 10 min at RT;
7. Discard the supernatant, flick the pellet and add another 10 ml of PBS;
8. Centrifuge at 250 rcf 10 min at RT;
9. Repeat steps 7 and 8;
10. Discard the supernatant and resuspend the pellet in 1 ml of complete medium (RPMI-1640 10% FCS);
11. Count cells and make a 4×10⁶ cell/ml suspension in complete medium. Add 100 μl of cell suspension per well in a V-bottom 96-well plate. Then add 50 μl of stimulant or vehicle in complete media, and 50 μl of drug or vehicle in complete media. Incubate the cells for 24 h at 37° C. 5% CO₂;
12. After incubation, take 60 μl of cell supernatant to measure IFNγ and TNFα by ELISA (OptEIA human IFNγ, cat No. 555142 and human TNF, Cat No. 555212) following manufacturer's instructions (BD Biosciences).

### LPS Stimulation Studies

The results of the LPS stimulation experiments are shown in Table 1. The values in the Table are expressed as the percentage value of the LPS only control. Thus, the maximum concentration of the cytokine, either IFN-γ or TNF-α, expressed from the PMBC cells in the presence of only LPS is said to be 100%, and the concentrations of the cytokines that are expressed from the PMBC cells in the presence of (i) LPS and (ii) ibuproxam (BC1048), benoxaprofen hydroxamate (BC1006) or pentoxifylline (BC1042), are expressed as a percentage of the LPS only 100% control. Standard deviation values (st error) are given underneath each value of expressed IFN-γ or TNF-α levels.

TABLE 1
Determination of IFN-γ and TNF-α levels under LPS stimulation
(Percentage IFN-γ and TNF-α levels compared to 100% untreated cells under
LPS stimulation)

| | | | IFN-γ | | | TNF-α | | |
|---|---|---|---|---|---|---|---|---|
| | | | 100 μM | 10 μM | 1 μM | 100 μM | 10 μM | 1 μM |
| BC1048 | LPS | % signal | −1.28 | 96.62 | 89.21 | 109.93 | 105.67 | 105.78 |
| | | st error | 1.33 | 19.86 | 9.78 | 2.39 | 2.35 | 1.32 |
| | no LPS | % signal | −6.29 | −3.67 | −4.14 | −1.28 | 0.48 | 3.14 |
| | | st error | 0.76 | 0.35 | 1.66 | 0.13 | 0.37 | 1.41 |
| BC1006 | LPS | % signal | 21.21 | 16.67 | 35.29 | 57.77 | 101.47 | 106.25 |
| | | st error | 4.21 | 3.12 | 11.08 | 3.96 | 0.30 | 3.40 |
| | no LPS | % signal | 12.38 | 13.46 | 16.74 | 11.97 | −2.27 | −2.29 |
| | | st error | 1.80 | 0.24 | 5.58 | 0.70 | 0.50 | 0.59 |
| BC1042 | LPS | % signal | 40.73 | 98.28 | 102.11 | 114.60 | 107.28 | 113.04 |
| | | st error | 20.56 | 13.60 | 42.65 | 2.05 | 1.37 | 1.51 |
| | no LPS | % signal | 2.03 | 1.48 | 3.98 | −0.80 | −2.45 | 1.15 |
| | | st error | 1.16 | 1.33 | 0.47 | 1.03 | 0.67 | 0.63 |

With reference to the data shown in Table 1, the inventors were surprised to observe that the concentrations of IFN-γ and TNF-α decreased in the presence of ibuproxam (BC1048), benoxaprofen hydroxamate (BC1006) or pentoxifylline (BC1042) in LPS stimulated cells. Ibuproxam completely blocks the production of IFN after stimulation at the highest concentration (100 M), while there is less of an effect on TNF. Benoxaprofen hydroxamate consistently inhibits the production of IFN (35% to 21%) at all concentrations used (1 to 100 M), and its maximal effect against TNF was at the higher concentration (41%). Pentoxifylline inhibited the production of IFN in a similar manner to ibuproxam, having an effect at 100 mM, although this effect was less pronounced than ibuproxam, and, again, less of an effect was seen against TNF.

### Con A Stimulation Studies

The results of the Con A experiments are illustrated in Table 2.

TABLE 2
Determination of IFN-γ and TNF-α levels under Con A stimulation
(Percentage IFN-γ and TNF-α levels compared to 100% untreated cells under Con A
stimulation)

| | | | IFN-γ | | | TNF-α | | |
|---|---|---|---|---|---|---|---|---|
| | | | 100 μM | 10 μM | 1 μM | 100 μM | 10 μM | 1 μM |
| BC1048 | ConA | % signal | 78.18 | 97.25 | 94.90 | 79.25 | 106.74 | 109.64 |
| | | st error | 12.67 | 0.87 | 1.70 | 9.32 | 2.97 | 1.85 |
| | no ConA | % signal | −1.04 | −0.61 | −0.68 | −1.25 | 0.48 | 3.08 |
| | | st error | 0.13 | 0.06 | 0.27 | 0.13 | 0.36 | 1.38 |
| BC1006 | ConA | % signal | 3.78 | 33.81 | 98.35 | 51.50 | 38.65 | 83.37 |
| | | st error | 0.60 | 5.36 | 0.31 | 8.61 | 12.73 | 11.24 |
| | no ConA | % signal | 1.82 | 2.80 | 3.76 | 19.51 | 1.62 | 2.84 |
| | | st error | 0.32 | 0.55 | 0.20 | 1.66 | 0.32 | 0.30 |
| BC1042 | ConA | % signal | 92.61 | 97.98 | 108.10 | 103.71 | 102.31 | 101.46 |
| | | st error | 10.71 | 5.92 | 2.65 | 4.18 | 1.58 | 0.87 |
| | no ConA | % signal | 0.58 | 0.42 | 1.13 | −0.77 | −2.37 | 1.11 |
| | | st error | 0.33 | 0.38 | 0.13 | 1.00 | 0.65 | 0.60 |

With reference to the data shown in Table 2, the inventors observed that the concentrations of TNF-α and IFN-γ also decreased in the presence of ibuproxam (BC1048), benoxaprofen hydroxamate (BC1006) or pentoxifylline (BC1042) in Con A stimulated cells. In this in vitro system, ibuproxam had a modest effect against ConA-stimulated IFN and TNF production at the highest concentration. Benoxaprofen hydroxamate had a greater effect versus IFN and TNF with clear effects at 10 and 100 M. Against this stimulus, pentoxifylline has little effect versus ConA-induced TNF or IFN production.

### SUMMARY

In summary, the inventors were surprised to observe that ibuproxam, oxametacin, benoxaprofen, benoxaprofen hydroxamate and pentoxifylline improve survival in influenza-challenged mice. They therefore believe that any compound represented by formula (I) or pentoxifylline may be used as an IFN-γ and TNF-α inhibitor, which can be used in the treatment of an infection with a pathogen which causes a respiratory disorder, such as influenza. The encouraging results of the in vivo mouse studies described in the Examples clearly demonstrate that mice infected with a H1N1 virus can be effectively treated by administration of a single dose of ibuproxam, oxametacin, benoxaprofen, benoxaprofen hydroxamate or pentoxifylline. Hence, it is clear that any compound (I) or pentoxifylline could be used to treat viral infections, or other pathogenic infections which causes a fulminant respiratory disorder.

1. A compound of formula I: 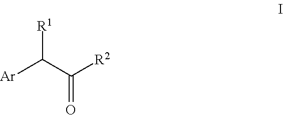
wherein,
Ar is an aryl or substituted aryl group,
R¹ is a C₁₋₃ alkyl group or hydrogen, and
R² is OH or —NHOH,
or a pharmaceutically acceptable salt, solvate, or solvate of a salt thereof.

2. The compound according to claim 1, wherein Ar is a substituted phenyl group.

3. The compound according to claim 1, wherein R¹ is hydrogen or a methyl group.

4. The compound according to claim 1, wherein R² is —NHOH.

5. The compound according to claim 1, wherein when Ar is a substituted phenyl group, the bond joining it to the remainder of the structure shown in formula I extends directly to a carbon atom in the phenyl ring.

6. The compound according to claim 1, wherein compound (I) is a 2-aryl, N-hydroxyacetamide, or 2-aryl, 2-methyl, N-hydroxyacetamide derivative.

7. The compound according to claim 1, wherein compound (I) is ibuproxam, oxametacin, benoxaprofen, or benoxaprofen hydroxamate.

8. The compound according to claim 1, wherein compound (I) is:

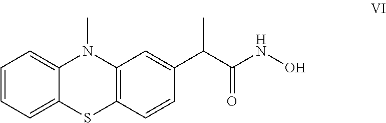 [SMILES: CC(C(=O)NO)c1ccc2c(c1)N(C)c1ccccc1S2]

9. The compound according to claim 1, wherein compound (I) is:

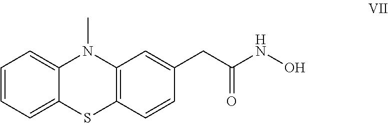 [SMILES: CN1c2ccccc2Sc2ccc(CC(=O)NO)cc21]

10-23. (canceled)

24. A pharmaceutical composition comprising a therapeutically effective amount of a compound, as defined in claim 1, and a pharmaceutically acceptable vehicle.

25. The pharmaceutical composition according to claim 24, wherein compound is pentoxifylline, ibuproxam, oxametacin, benoxaprofen, benoxaprofen hydroxamate, or a pharmaceutically acceptable salt, solvate or solvate of a salt thereof.

26. The pharmaceutical composition according to claim 24, wherein the compound is:

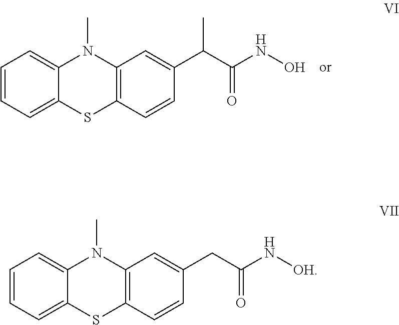 [SMILES: CC(C(=O)NO)c1ccc2c(c1)N(C)c1ccccc1S2 ; CN1c2ccccc2Sc2ccc(CC(=O)NO)cc21]

27. A method of treating an infection, the method comprising the step of administering, to a subject in need of such treatment, a therapeutically effective amount of a compound as defined in claim 1, wherein the infection causes a respiratory disorder, and wherein administration of the compound ameliorates a symptom associated with the infection, thereby treating the subject.

28. The method according to claim 27, wherein compound is pentoxifylline, ibuproxam, oxametacin, benoxaprofen, benoxaprofen hydroxamate, or a pharmaceutically acceptable salt, solvate or solvate of a salt thereof.

29. The method according to claim 27, wherein the compound is:

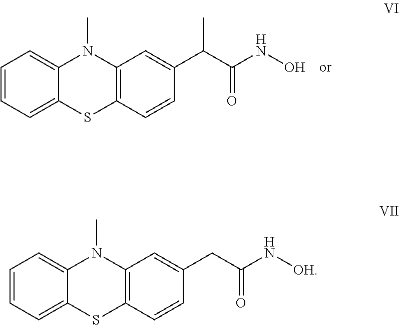 [SMILES: CC(C(=O)NO)c1ccc2c(c1)N(C)c1ccccc1S2 ; CN1c2ccccc2Sc2ccc(CC(=O)NO)cc21]

30. The method according to claim 27, wherein the infection is a common cold, a sinusitis, a tonsillitis, an otitis media, a pharyngitis, a laryngitis, a pneumonia, a Respiratory Distress Syndrome (RDS), an Acute Respiratory Distress Syndrome (ARDS), or an Acute Lung Injury (ALI).

31. The method according to claim 27, wherein the infection is a bacterial infection, a fungal infection or a viral infection.

32. The method according to claim 31, wherein the viral infection is caused by a paramyxovirus or an orthomyxovirus infection.

33. The method according to claim 31, wherein the viral infection is caused by an Influenzavirus A serotype, an Influenzavirus B serotype, an Influenzavirus C serotype, or a derivative thereof.

34. The method according to claim 33, wherein the Influenzavirus A serotype is H1N1, H1N2, H2N2, H3N1, H3N2, H3N8, H5N1, H5N2, H5N3, H5N8, H5N9, H7N1, H7N2, H7N3, H7N4, H7N7, H9N2, H10N7, or a derivative thereof.

35. The method according to claim 33, wherein the Influenzavirus A serotype is H1N1, or a derivative thereof.

36. The method according to claim 27, wherein administration of the compound modulates IFN-γ and/or TNF-α.

37. The method according to claim 27, wherein the symptom comprises an inflammatory symptom of a virally-induced cytokine production.

38. The method according to claim 27, wherein the symptom comprises an inflammatory symptom of an acute viral infection.

39. The method according to claim 27, wherein the symptom comprises an inflammatory symptom of a viral flare-up.
